(19) United States
(12) Patent Application Publication
(10) Pub. No.: US 2022/0031198 A1
(43) Pub. Date: Feb. 3, 2022

(54) SURGICAL MUSCLE PARALYSIS MEASUREMENT DEVICE

(71) Applicant: Worcester Polytechnic Institute, Worcester, MA (US)

(72) Inventors: Christopher R. Beauregard, Litchfield, NH (US); Nicholas R. Bergstrom, Laconia, NH (US); Edward G. Crofts, Worcester, MA (US); Kyler B. Dillon, Cranston, RI (US); Anastasia Karapangou, Worcester, MA (US); Kinsey McNamara, Chelmsford, MA (US); Ahmet C. Sabuncu, Ashland, MA (US); Eric Rosero, Dallas, TX (US)

(21) Appl. No.: 17/501,516

(22) Filed: Oct. 14, 2021

Related U.S. Application Data
(63) Continuation of application No. PCT/US2020/028442, filed on Apr. 16, 2020.
(60) Provisional application No. 62/834,723, filed on Apr. 16, 2019.

(51) Int. Cl. A61B 5/11

(52) U.S. Cl. CPC A61B 5/1106; A61B 2562/168; A61B 2562/0247

(57) ABSTRACT

A neuromuscular test device for assessing a level of neuromuscular blocking agents (NMBAs) in an anesthetized patient includes a stimulation circuit for initiating a muscular stimulus in a muscle structure, and a testing circuit for measuring an activity level responsive to the muscular stimulus. A flexible, closed vessel is responsive to a musculature response for inducing a pressure, and a pressure sensor in communication with the closed vessel generates a pressure signal indicative of the musculature response to the muscular stimulus.

### BACKGROUND

During surgery, neuromuscular blocking agents (NMBAs) are administered to temporarily paralyze the patient and allow for endotracheal intubation. Monitoring is performed throughout the process to ensure that the neuromuscular blockade is sufficient for the surgery to proceed and that it has sufficiently diminished to allow for extubation without residual blockade. One potential issue that physicians need to monitor for is that residual neuromuscular blockade can lead to ICU-acquired weakness and in some cases critical respiratory issues.

Conventional approaches for measuring muscle paralysis for surgical anesthesia include kinemyography, which measures the strain of a piezoelectric strip on the skin, and electromyography, which measures the action potential of a muscle. These devices have been shown to overestimate the neuromuscular recovery, which leads to inaccurate results and therefore an underdose of neuromuscular blockers given to the patient. Mechanomyography (MMG) may be a more reliable measurement, however the MMG is not used in devices due to limitations in mounting and stabilization. Acceleromyography (AMG), which measures the acceleration of a part of the body, has been used in practice but has significant reliability limitations.

### SUMMARY

A neuromuscular test device for assessing a level of neuromuscular blocking agents (NMBAs) in an anesthetized patient includes a stimulation circuit for initiating a muscular stimulus in a muscle structure, and a testing circuit for measuring an activity level responsive to the muscular stimulus. A flexible, closed vessel is responsive to a musculature response for inducing a pressure, and a pressure sensor in communication with the closed vessel generates a pressure signal indicative of the musculature response to the muscular stimulus.

Anesthesiologists monitor a patient's level of intraoperative neuromuscular blockade for identifying an appropriate level of anesthesia for patient administration. During surgery, neuromuscular blocking agents (NMBAs) are administered to temporarily paralyze the patient and allow for endotracheal intubation. Monitoring is required throughout the process to ensure that the neuromuscular blockade is sufficient for the surgery to proceed and that it has sufficiently diminished to allow for extubation without residual blockade. One potential issue that physicians need to monitor for is that residual neuromuscular blockade can lead to ICU-acquired weakness and in some cases critical respiratory issues.

A standard way of quantitatively measuring neuromuscular blockade is called Train-of-Four (TOF). TOF involves the use of a peripheral nerve stimulator (PNS) which provides a TOF electrical impulses to the patient's ulnar nerve through electrodes placed on the skin. These impulses stimulate the nerve, producing a response in the form of a twitch in the muscle. The response of the associated muscle to this stimulus is then measured via discrete quantity of twitches (e.g. 2 out of 4 twitches) or a ratio of the amplitude of the last twitch to that of the first (e.g. 0.8 or 80%). The TOF Ratio considers the fade caused by a nondepolarizing NMBA. Fade is the decrease in amplitude between the first and final twitches in a TOF. This ratio is a key metric used to determine readiness for extubation and is difficult to evaluate without the use of a quantitative measuring device.

Configurations herein are based, in part, on the observation that the TOF stimulation is a common approach for quantitatively measuring neuromuscular blockade. Unfortunately, conventional approaches to TOF measurement suffer from the shortcoming that formal measurement devices for TOF response are often not available due to expense and/or complexity. Instead, subjective measurement techniques such as manual observation are employed. Accordingly, configurations herein substantially overcome the above-described shortcomings of TOF feedback by providing a low cost, simple yet effective measurement that relies upon compressive air pressure resulting from digit movement. The quantitative measurement of air forced from a closed, flexible vessel allows measurement of musculature response from movement in any axis and standardizes the magnitude of the sensed response.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a block diagram of configurations herein;

FIG. 2 shows a thumb wedge arrangement of the musculature feedback element of FIG. 1;

FIG. 3 shows a palm arrangement of the musculature feedback element of FIG. 1;

FIG. 4 shows a thumb compression arrangement of the musculature feedback element of FIG. 1;

FIG. 5 shows a mounting arrangement for the pressure sensor of FIG. 1; and

FIG. 6 shows a full system implementation on a patient's wrist/hand as depicted in FIG. 1.

### DETAILED DESCRIPTION

Configurations below demonstrate an approach to TOF response monitoring for NMBA quantification. Because none of the conventional TOF measuring techniques are usable with immobile hands, the disclosed approach was developed. This technique relies on the capture of limited movement of the thumb. It also provides the capture of varying motion along multiple axes. Thus, the physical interface captures small, multidirectional displacements of the thumb. The solution includes a variation on a flexible vessel such as a bulb-pump. The physical output is a pressure produced by the deflection of a flexible balloon closed at one end with tubing attached to the other. The pressure is transmitted through the tubing where it will reach a pressure sensor of transducer which converts the pressure to an electrical potential. This allows for the measurement of very small thumb movements to demonstrate an anesthetization level.

In order to capture movement in multiple axes, the combination of the shape of the balloon and the resting position of the thumb (dictated by the mounting system) should make possible movements of the thumb produce a force on the balloon which is normal to its surface. This would allow proper transmission of pressure through the balloon. Another important aspect of the design is that the balloon must be constrained such that the force of the thumb on the balloon results in pressure transmission rather than displacement of the balloon.

In conventional approaches, sensing mediums such as kinemyography and electromyography may be employed. Kinemyography measures the strain of a piezoelectric strip on the skin while electromyography measures the action potential of a muscle. These devices have been shown to overestimate the neuromuscular recovery. This leads to inaccurate results and therefore an underdose of neuromuscular blockers given to the patient. While mechanomyography (MMG) is acknowledged as the most reliable measurement for train of four, the MMG is not used in devices due to limitations in mounting and stabilization. Acceleromyography (AMG), which measures the acceleration of a part of the body, has been used but has significant reliability limitations. It has been shown that data collected using these devices varied between tests. The disclosed use of the pressure sensor eliminates the variation between tests and is reliable while maintaining the ability to be stabilized and avoid significant noise.

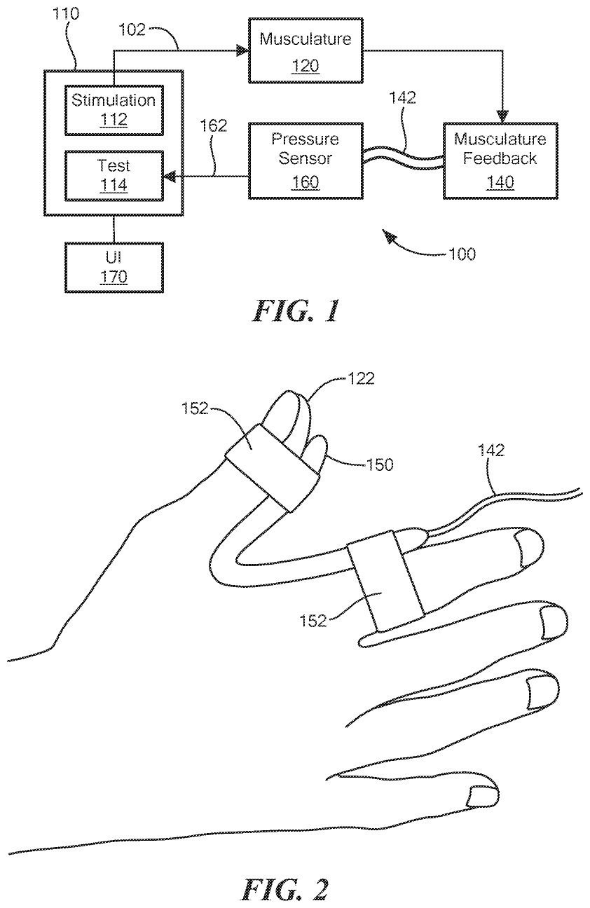

FIG. 1 shows a block diagram of configurations herein. Referring to FIG. 1, in an anesthesia environment 100 relying on neuromuscular activity feedback for assessing an effective level of anesthetization, a neuromuscular test device 110 includes a stimulation circuit 112 for initiating a muscular stimulus 102 in a muscle structure 120 (musculature), and a testing circuit 114 for measuring an activity level responsive to the muscular stimulus. A musculature feedback 140 is captured by a flexible, closed vessel, discussed further below, responsive to a musculature response for inducing a pressure. A pressure sensor 160 is in communication with the closed vessel for generating a pressure signal 162 indicative of the musculature response to the muscular stimulus. The pressure signal is generally based on a magnitude of a fluid (i.e. air) forced though a fluidic pressure medium 142, forced from compression and/or movement from the musculature feedback 140 response. A User Interface (UI) 170 such as a standard video and keyboard arrangement provides user commands and feedback.

FIG. 2 shows a thumb wedge arrangement of the musculature feedback element of FIG. 1. Referring to FIGS. 1 and 2, the stimulation circuit 112 induces a response in a thumb 122 or other digit in the musculature 120 responsive to the stimulation circuit. 112. The stimulation circuit 112 typically attaches to two location (discussed further below) on the forearm for cathode and anode locations selected to induce a response in the musculature 120 by an applied voltage/current.

The closed vessel 150 takes the form of a “V” shaped balloon for providing musculature feedback 140 as the thumb 122 closes against the adjacent digits and/or the palm. In various configurations discussed below, the closed vessel 150 is a balloon structure engaged with a digit and adapted to compress in response to movement of the digit from the muscular stimulus. Straps 152 secure the closed vessel 150 in engagement with the thumb 122 and/or other digits such that compressive action forces air through the pressure medium 142, such as a flexible tube. Since any compression of the closed vessel 150 will induce a pressure flow in the pressure medium 142, the thumb movement can be in a variety of directions and need not meet or “pinch” the index finger along any particular axis, for example, in order for providing the musculature feedback 140.

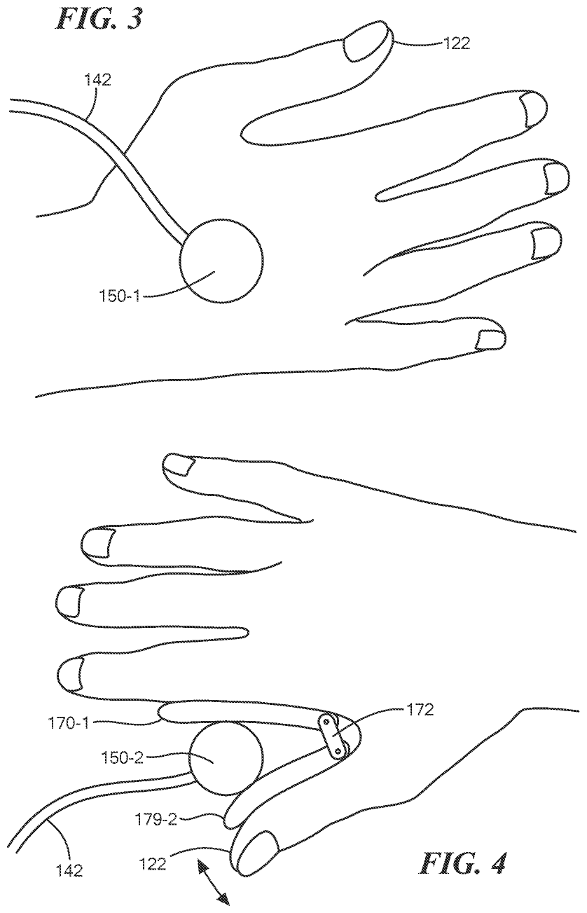

FIG. 3 shows a palm arrangement of the musculature feedback 140 element of FIG. 1. Referring to FIGS. 1 and 3, an alternate closed vessel 150-1 is disposed on the palm for reengagement by the thumb or other digit/movement. Straps, adhesive or gravity, or a combination thereof, may serve to keep the closed vessel 150-1 in place for providing musculature feedback 140.

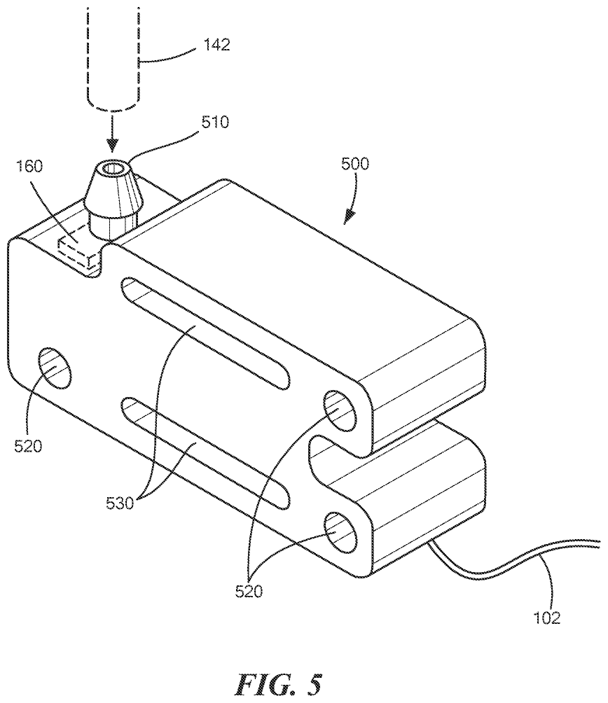

FIG. 4 shows a thumb compression arrangement of the musculature feedback element of FIG. 1. Referring to FIGS. 1 and 4, a pair of hinged plates 170-1 . . . 170-2 (170 generally) pivot about a hinge 172. The hinged plates 170 flank the closed vessel 150-2 for compressing the closed vessel 150-2 and inducing a pressure from thumb 122 (or other digit) movement.

FIG. 5 shows a mounting arrangement for the pressure sensor of FIG. 1. Referring to FIGS. 1-5, the pressure sensor 160 receives the pressurized air or other gas from the closed vessel 150 and generates a pressure signal 162 to the test circuit 114. A fluidic connection from pressure medium 142 between the closed vessel 150 and the pressure sensor 160 disposes the pressure sensor more proximate to the closed vessel 150 than to the testing circuit 114. Reduction of the distance that the fluidic pressure medium 142 carries the pressurized air serves to mitigate any deviation that may arise from characteristics of a tube or other medium defining the fluidic pressure medium. The electronic pressure signal 162 may then be carried by wires to the test circuit 114.

A wrist frame 500 or other remotely mountable support for the pressure sensor 160 allows physically sensed pressure to occur as close as possible to the closed vessel 150. The wrist frame 500 includes a pressure inlet 510 for coupling with tubing defining the fluidic pressure medium 142. Fastener apertures 520 engage screws or pegs for securing the frame 500, and strap holes 530 allow for wrist straps to dispose the frame 500 in close proximity to the closed vessel 150.

In the example configuration, an expected range of the pressure signal is between 0.2-2 psig. The pressure sensor is an analog sensor having a range of 0-5 psig, and an accuracy of ±0.0125 psi, making it sensitive enough to record subtle variations in the pressure generated during a train of four test. Pressure values are updated at approximately 1 kHz for this sensor, and the sensor is calibrated to work between 0° C. and 50° C. and can be used with dry gases such as ambient air. Any suitable pressure sensor may be employed, and variances such as digital sensing, finer pressure granularity and update frequencies may be employed.

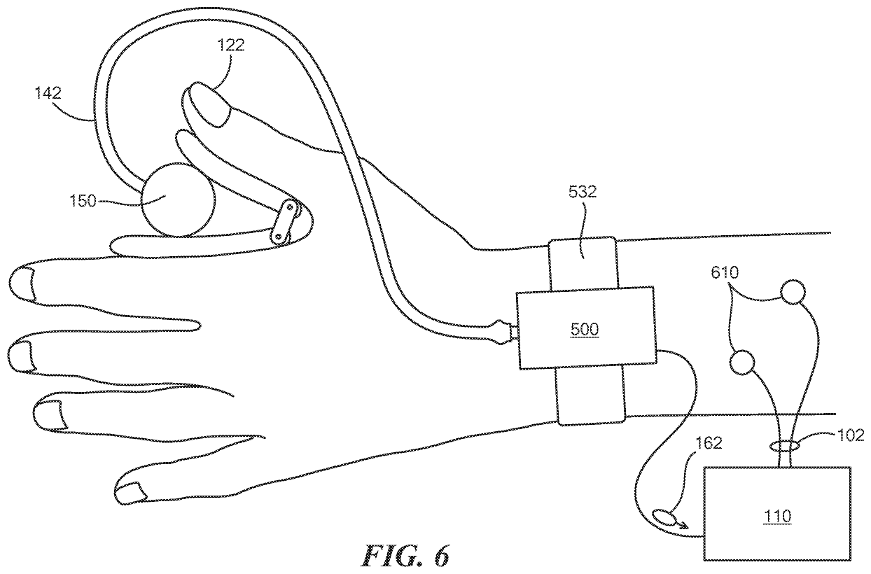

FIG. 6 shows a full system implementation on a patient's wrist/hand as depicted in FIG. 1. Operation includes obtaining a musculature response resulting from the stimulation circuit 112, and quantifying this response via the testing circuit 114. For proper nerve stimulation, the chosen nerve must meet basic criteria such as containing a motor element, being close to the surface, and producing a visible muscle contraction. The testing circuit 114 includes a plurality of electrodes adapted for electrical coupling to the muscle structure. Referring to FIGS. 1-6, to achieve nerve stimulation, an electric current is supplied through the skin using electrocardiogram (ECG) electrodes 610. The PNS is calibrated for each patient using a test performed while the patient is fully conscious. The ideal nerve stimulator should provide controllable, yet constant current since its magnitude affects whether a nerve is activated. The current also should be no greater than 80 mA, as currents above this are unsafe to use. The resistance of a patient's skin is generally between 0-5 kΩ Most nerve stimulators supply monophasic, square wave with a period of 0.1-0.3 ms because there is no need for a greater time interval than this to produce accurate results. The electrodes 610 include a negative electrode and a positive electrode, in which the negative electrode should be placed over the most superficial part of the nerve, while the positive electrode should be placed along the course of the nerve. For TOF twitch simulation, a frequency of 2 Hz with at least 10 seconds between trains is preferable.

From a biomechanics standpoint, it is desirable to maximize the moment of the thumb around the joint which is quantified as the cross product of the distance and the force of the thumb twitch. With a larger distance vector, the moment created is also larger which allows for a larger force being translated into the balloon. Thus, the thumb 122 should have the greatest distance possible to the balloon and the tip of the thumb should strike the balloon. This would occur at the proximal phalanx for the most natural twitch motion.

In operation, the area of the arm where the electrodes 610 are placed is cleaned with alcohol and shaved whenever necessary. The electrodes were placed on the forearm. These may be standard ECG electrodes that are used on a regular basis in hospitals. The PNS provides a train-of-four electrical impulses to the nerve of the thumb through the electrodes placed on the skin of the arm. The PNS is calibrated to each participant by first selecting a zero amplitude and then, slowly increasing in increments of 10 mA until 4 twitches in the thumb was observed. The current was not greater than 80 mA to ensure safe current levels.

The stimulation 112 and test circuits 114 may be fulfilled by any suitable electronic device operable for delivering the muscular stimulus 102 to the electrodes 610 and for receiving and analyzing the pressure signal 162.

Configurations herein identify a pressure response from the gas in the closed vessel based on the musculature feedback 140 from the partially anesthetized muscle. Due to limitations in the sensitivity and precision of pressure sensors and/or transducers, the change in pressure created from the deformation of and subsequent flow in the fluidic pressure medium 142 is identified and correlated to musculature activity. To provide a cursory understanding of the relationships between geometric parameters and pressure output, a simple two-dimensional model is employed. In an example herein, the testing circuit 114 implements computations of an effect of the muscular contractions based on Hertzian contact of deformable bodies and adiabatic compression of an ideal gas.

Computations based on the received pressure signal 162 benefit by close adherence to several ideal conditions, including:

The contacting bodies are isotropic and elastic;

The contact areas are essentially flat and small relative to the radii of curvature of the undeformed bodies in the vicinity of the interface; and

The contacting bodies are perfectly smooth, and therefore only normal pressures are taken into account.

The bodies in question can be considered elastic, and smooth, but may not be isotropic radially. They are, however, isotropic in the circumferential direction. The second assumption is somewhat variable due to the large deformation of the balloon. Hertzian stresses are, therefore, not a perfectly accurate model of the system, but can provide relationships between parameters and outputs e.g., the relationship may be that pressure decreases quadratically with radius, and Hertz gives a linear relationship, however we gain an understanding that increased radius leads to decreased pressure.

Hertzian analysis was carried out for a spherical surface assumed to be in contact with a flat surface (spherical surface was chosen due to the limitations of Hertzian contact analysis in obtaining the deflection of cylindrical contact with flat surfaces), as per the following:

$$\begin{matrix}a = 0.880 \sqrt[3]{F r Δ} & ( 1 ) \\ δ = 0.775 \sqrt[3]{\frac{{F}^{2} {Δ}^{2}}{r}} & ( 2 )\end{matrix}$$

Where: a is the radius of contact, r is the radius of the sphere, F is the applied force, δ is the deflection of the sphere, and:

$$Δ = \frac{1}{{E}_{1}} + \frac{1}{{E}_{2}}$$

Where E₁, and E₂ are the elastic modulus of the sphere and flat surface respectively. The modulus of the sphere was taken as the bulk modulus of the entire balloon. The bulk modulus of a fluid filled spherical shell may be obtained by:

$${k}_{b} = f + \frac{( \frac{4 t}{3 b} ) g ( 1 + v )}{1 - v} \qquad ( 3 )$$

where f is the fluid bulk modulus, g is the shear modulus of the solid (walls), and v is the Poisson's ratio of the solid. Then we have:

E=3k_{b}(1−2v) (4)

Using these results, the simple model assumed that the sphere was deflected instantaneously by the applied force to a deformed state which, for the sake of simplicity, was modeled as a sphere with a cap of height, δ, removed. Therefore, the change in volume was found to be:

$$Δ V = \frac{π}{3} [ {δ}^{2} ( 3 r - δ ) ] \qquad ( 5 )$$

The process was assumed adiabatic, thus the change in pressure due to the initial deformation was obtained as:

$$Δ P = \{ {[ \frac{{V}_{1}}{{V}_{1} + Δ V} ]}^{γ} - 1 \} {P}_{1} \qquad ( 6 )$$

where the heat capacity ratio is defined as:

$$γ = \frac{{C}_{p}}{{C}_{v}}$$

The balloon was then assumed a sphere of volume V₁+ΔV. The pressure change was then modeled to instantaneously create a dilation of the sphere as described by:

$$\begin{matrix}δ = \frac{Δ {Pr}^{2}}{2 hE} - ( 1 - v ) & ( 7 ) \\ Δ V = \frac{4 π}{3} [ {( r + δ )}^{3} - {r}^{3} ] & ( 8 )\end{matrix}$$

Finally, a final change in pressure was obtained using equation (6). For each parameter, if the domain is large enough, the model predicts an optimal value can be chosen to maximize the pressure. If a custom balloon were to be designed and manufactured, optimal parameters may be chosen.

The above computations may be performed by any suitable processing device, circuit and/or state machine for implementing instructions based on the equations, such as an ARDUINO® or RASPBERRY PI®, optionally coupled to a more substantial processing device for statistical or other analysis.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. In an anesthesia environment relying on neuromuscular activity feedback for assessing an effective level of anesthetization, a neuromuscular test device, comprising:
a fluidic coupling between a mechanical actuation and a pressure sensor responsive to a flow through the fluidic coupling;
a stimulator for initiating a muscular stimulus for triggering the mechanical actuation via a neuromuscular response; and
a testing circuit responsive to measure the flow based on the triggered mechanical activation.

2. The device of claim 1 wherein the testing circuit has a processor for computing a magnitude of the neuromuscular response based on the measured flow.

3. The device of claim 1 wherein the mechanical activator is a closed, flexible vessel responsive to compression from an actuated musculature.

4. The device of claim 1 wherein the mechanical actuation is a pressure response from a compressed vessel.

5. The device of claim 4 further comprising a hinged structure having opposed plates, the opposed plates responsive to the mechanical actuation for pivotal movement of the hinge for compressing a vessel between the opposed plates for inducing the flow.

6. The device of claim 1 further comprising a flexible, closed vessel disposed in a path of the mechanical actuation, the closed vessel having a volume responsive to displacement from compression resulting from the mechanical actuation, the flow driven by the displaced volume.

7. The device of claim 1 further comprising an elongated, articulated vessel, the elongated articulated vessel formed of a flexible material and having a fluidic volume responsive to external compression, the elongated, articulated vessel disposed between pivoting members and responsive to compression from the pivoting members for expelling the fluidic volume.

8. The device of claim 3 further comprising:
a fluidic vessel between a sensing frame and the vessel, the sensing frame having a receptacle for engaging the fluidic vessel;
the sensing frame enclosing the testing circuit and adapted for engaging a strap for securement to a testing site; and
a sensing element in the sensing frame, the sensing element coupled to the receptacle for measuring the flow.

9. A method for measuring neuromuscular activity, comprising:
disposing a pressure responsive fluidic volume in a path of mechanical travel;
transmitting a stimulation signal to a musculoskeletal structure, the musculoskeletal structure configured for responsive movement along the path; and
measuring a fluidic flow in response to the movement if the musculoskeletal structure along the path for determining a magnitude of a neuromuscular response.

10. The method of claim 9 wherein the responsive movement along the path results in an interference with a fluidic vessel, the interference causing compression of the fluidic vessel for inducing the fluidic flow.

11. The method of claim 9 further comprising computing, in a testing circuit having a processor, the magnitude of the neuromuscular response based on the measured flow.

12. The method of claim 9 further comprising enclosing the fluidic volume in a closed, flexible vessel responsive to compression from the movement of the musculoskeletal structure.

13. The method of claim 12 wherein measuring the fluidic flow further comprises receiving a pressure response from a compressed vessel.

14. The method of claim 12 wherein the vessel further comprises an elongated, articulated vessel, the elongated articulated vessel formed of a flexible material and having a fluidic volume responsive to external compression, further comprising compressing the elongated, articulated vessel disposed between pivoting members for expelling the fluidic volume.

15. The method of claim 9 further comprising actuating a hinged structure having opposed plates, the opposed plates responsive to the responsive movement for pivotal movement of the hinge for compressing a vessel between the opposed plates for inducing the fluidic flow.

16. In an anesthesia environment relying on neuromuscular activity feedback for assessing an effective level of anesthetization, a neuromuscular test device, comprising:
a stimulation circuit for initiating a muscular stimulus in a muscle structure;
a testing circuit for measuring an activity level responsive to the muscular stimulus;
a flexible, closed vessel responsive to a musculature response for inducing a pressure; and
a pressure sensor in communication with the closed vessel for generating a pressure signal indicative of the musculature response to the muscular stimulus.

17. The device of claim 16 wherein the closed vessel is a balloon structure engaged with a digit and adapted to compress in response to movement of the digit from the muscular stimulus.

18. The device of claim 16 further comprising a fluidic connection between the closed vessel and the pressure sensor, the fluidic connection for disposing the pressure sensor more proximate to the closed vessel than the testing circuit.

19. The device of claim 16 wherein the testing circuit includes a plurality of electrodes adapted for electrical coupling to the muscle structure.
